(19) United States
(12) Patent Application Publication
(10) Pub. No.: US 2023/0372561 A1
(43) Pub. Date: Nov. 23, 2023

(54) APPARATUS AND METHOD FOR UV-C MASK SANITIZATION

(71) Applicant: FLORIDA ATLANTIC UNIVERSITY BOARD OF TRUSTEES, Boca Raton, FL (US)

(72) Inventor: Patrick A. GRANT, Boca Raton, FL (US)

(21) Appl. No.: 18/030,315

(22) PCT Filed: Sep. 28, 2021
(86) PCT No.: PCT/US2021/052383

§ 371 Date: Apr. 5, 2023

Related U.S. Application Data
(60) Provisional application No. 63/087,488, filed on Oct. 5, 2020.

(51) Int. Cl. A61L 2/10

(52) U.S. Cl. CPC A61L 2/10; A61L 2202/11; A61L 2202/122

(57) ABSTRACT

An apparatus and method for sanitizing contaminated masks with UV-C radiation in a home or work environment is disclosed. The apparatus includes a rack structure with a plurality of arms, wherein each arm contains a fastener to permit users to secure an N-95, cloth, or surgical mask to the fastener in an expanded position. A UV-C bulb of sufficient size to provide a sanitizing effect yet prevent significant emission of ozone is placed on the rack structure to deliver uninterrupted UV-C rays to the mask surfaces. A housing structure covers the entire unit, including an inner reflective metallic surface and forming a seal to prevent the escape of UV-C radiation from the housing structure. A timing mechanism controls the length of time to which masks are exposed to UV-C light and alerts the user when sanitization is complete. An interlock mechanism prevents bulb activation unless the rack/bulb are covered.

### CROSS-REFERENCE TO RELATED APPLICATIONS

This PCT Application claims the benefit of 35 U.S.C. § 119(e) of Application Ser. No. 63/087,488, filed on Oct. 5, 2020 entitled APPARATUS AND METHOD FOR UV-C SANITIZATION OF CONTAMINATED MASKS IN A HOUSEHOLD OR WORKPLACE ENVIRONMENT and whose entire disclosure is incorporated by reference herein.

### BACKGROUND OF THE INVENTION

The present invention is related generally to sanitization utilizing ultraviolet light, more particularly an apparatus and method for sanitizing surgical, cloth, N-95, or other masks with UV-C radiation in a home or workplace environment.

Face coverings, such as surgical, cloth, or N-95 masks, have long been regarded as one of the most effective ways to prevent the spread of pathogens from person to person [1]. Mask usage may be traced back to seventeenth century Europe, where physicians commonly wore beak-like facial coverings filled with spices designed to neutralize pathogens in the air [2]. In the nineteenth century, painters and craftsmen wore facial coverings to protect their airways from dust and harmful particles [2]. The usage of facial coverings carried into the twentieth century where the advent of the disposable surgical mask revolutionized sanitary medical practices, preventing infections by precluding medical staff's pathogen-containing respiratory droplets from entering patients' open wounds [2].

Particularly, masks have been utilized during pandemics to prevent the spread of respiratory illness. The global spread of SARS-CoV-2, the virus known to cause COVID-19 in patients, has increased the rate of daily mask usage amongst the public beyond that previously experienced during the 1918 flu pandemic [3]. Use of facial coverings may both protect others from the wearer's respiratory droplets and protect the wearer from contaminants in the airspace [3,4]. With the widespread demand for masks and need to preserve personal protective equipment for those in the healthcare industry, a shortage in mask supply has led many to reuse masks or other facial coverings multiple times without sanitizing them [3].

Further, there is a need to minimize environmental pollution from the disposal of single-use masks. As traditional surgical masks are often discarded after one use, the amount of waste attributable to disposable masks has increased, creating a need for the public to be able to sanitize and reuse masks.

Mask sanitization is imperative for safe and effective usage [5, 6]. After each wear, bacteria from even a healthy wearer's own respiratory droplets collect on the inside of the mask, and the outside of a mask may potentially contain airborne pathogens capable of living on its surface [5]. The Center for Disease Control (CDC) has advised that masks be sanitized regularly, ideally recommending cleaning after every wear to prevent spread of disease [6, 7]. However, individuals only have access to a limited supply of masks and often do not have the option of disposing of the masks after a single use [3]. Those who wear cloth masks may sanitize them by washing them, but the washing and drying process is often too time-consuming to sustain washing after each wear [6]. Further, washing is not an option for those who wear medical-grade masks, and using disinfectant sprays can cause skin irritation or damage the fibers of the mask designed to catch particulates [6,7]. Thus, there is a need for a time-efficient method to sanitize facial coverings without damaging their effectiveness and enable wearers to safely reuse them in their daily lives.

Studies show the efficacy of ultraviolet (UV) radiation in sanitization [8]. The short-wavelength radiation is capable of destroying the nucleic acids present in microorganisms' genetic material, deactivating their pathogenic qualities [8,9]. Ultraviolet-C (UV-C) light has been used for sanitizing medical instruments, disinfecting rooms, and purifying air and water through filtration devices [8, 9, 10]. Examples of commercially-available UV-C treatment devices are: Prescientx's Terminator CoV system, masOd's Sanitizing Case, MegaVolt's Germicidal Charging Station, UVFAB's TrueClean-400 system, Atomic Blue Group's INFO Germicidal UV Light, and CaptureTech's CapCleaner UV-C Chamber. However, many of the devices permitting sanitization of multiple objects are of a commercial size too large for household use [9]. These devices frequently utilize larger UV bulbs, which are not only costly, but also produce significant amounts of ozone, yielding an unpleasant odor [10].

Further, current devices of a portable size are not suitably adapted to sanitize the entire surface of multiple masks at once, a concern for families using multiple face coverings on a daily basis. While handheld-UV wands exist, these wands increase exposure to UV rays and often require the user to hold the wand for a lengthy period of time to properly sanitize the desired surface, increasing the user's risk of skin burns and damage to the corneas of the eyes [11, 12]. Studies have shown users of hand-held devices are unable to hold the devices at the angle and for the length of time necessary to generate a stable UV directional output and effectively sanitize a surface [13]. Moreover, open-air UV devices risk UV exposure to commonly found household surfaces such as plastics, which may damage the integrity and appearance of these materials [14]. Thus, there remains a need for an apparatus to effectively sanitize masks that is safe, affordable, odor-free, and suitable for household use.

All references cited herein are incorporated herein by reference in their entireties.

### BRIEF SUMMARY OF THE INVENTION

An apparatus for sanitizing masks with UV-C radiation in a household or workplace environment is disclosed. The apparatus comprises: a rack structure having a plurality of arms, wherein the arms comprise fasteners capable of securing masks in an expanded position; a light source (e.g., a low-pressure mercury bulb or a light emitting diode (LED) bulb) capable of emitting UV-C radiation, wherein the light source is positioned to deliver continuous UV-C rays (e.g., 253.7 nm) to the mask surfaces for a predetermined period (e.g., 5 minutes) and is of a bulb size and wavelength to effectively sanitize a mask yet prevent significant emission of ozone; a lamp base stabilizes the light source and serves as a point of connection for the rack structure; a closed, removable housing structure, wherein the housing structure covers the rack and UV-C light source so as to prevent UV-C light from exiting the apparatus during UV-C light source activation, wherein the inner surface of the housing structure comprises a metallic surface capable of reflecting UV-C light within the apparatus.

A timer may also be included for allowing an operator to set the predetermined period (e.g., 5 minutes) for activating the UV-C light source for sanitizing the contaminated masks and then de-activating the UV-C light source when sanitization is complete.

An interlock may also be provided that prevents activation of the UV-C light source unless the housing structure is covering the rack structure and the UV-C light source.

A method for UV-C sanitization of contaminated masks in a household or workplace environment is also disclosed. The method comprises: providing a rack structure, configured for holding a plurality of contaminated masks, with a UV-C light source (e.g., a low-pressure mercury bulb or a light emitting diode (LED) bulb) positioned within the rack structure; covering the rack structure with a housing structure to reflect UV-C radiation (e.g., 253.7 nm) that emanates from the UV-C light source, when activated, while also protecting any one in a vicinity of the UV-C light source from exposure to the UV-C radiation; activating the UV-C light source to deliver continuous UV-C radiation for a predetermined period (e.g., 5 minutes) towards the plurality of contaminated masks in order to sanitize the plurality of contaminated masks; deactivating the UV-C light source; and providing access to sanitized masks via the housing structure.

The activation step may include setting a timer by an operator to ensure the predetermined period is implemented properly in the method.

The activation step may also include an interlock that prevents activation of the UV-C light source from activating unless the housing structure is covering the rack structure and the UV-C light source.

### BRIEF DESCRIPTION OF SEVERAL VIEWS OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a view of the rack structure of an embodiment of the apparatus of the present invention;

FIG. 2 is a side view of the length of the rack structure of an embodiment of the apparatus of the present invention;

FIG. 3 is a top view of the rack structure of an embodiment of the apparatus of the present machine;

FIG. 4 is a side view of the width of the rack structure of an embodiment of the apparatus of the present invention;

FIG. 5 is a side view of the length of the rack structure of an embodiment of the apparatus of the present invention, wherein the embodiment of the apparatus hangs two masks;

FIG. 6 is a top view of the inner structure of an embodiment of the apparatus of the present invention, wherein the embodiment of the apparatus hangs two masks;

FIG. 7 is an angled top view of the width of the rack structure of an embodiment of the apparatus of the present invention, wherein the embodiment hangs six masks;

FIG. 8 is a side view of the width of the rack structure of an embodiment of the apparatus of the present invention, wherein the embodiment hangs six masks;

FIG. 9 is a side view of the width of the rack structure of an embodiment of the apparatus of the present invention, wherein the embodiment hangs six masks;

FIG. 10 is an angled side view of the length of the rack structure of an embodiment of the apparatus of the present invention, wherein the embodiment is capable of hanging six masks;

FIG. 11 is a top view of the rack structure of an embodiment of the apparatus of the present invention, wherein the embodiment hangs six masks;

FIG. 12 is a top view of the rack structure of an embodiment of the apparatus of the present invention, wherein masks are visible on three of the six arms of the apparatus;

FIG. 13 is a side view of the width of the rack structure of an embodiment of the apparatus of the present invention, wherein masks are visible on three of the six arms of the apparatus;

FIG. 14 is a side view of the length of the rack structure of an embodiment of the apparatus of the present invention, wherein masks are visible on three of the six arms of the apparatus;

FIG. 15 is a top view of the rack structure of an embodiment of the apparatus of the present invention, wherein masks are visible on three of the six arms of the apparatus;

FIG. 16 is a side view of the rack structure of an embodiment of the apparatus of the present invention, wherein masks are visible on three of the six arms of the apparatus;

FIG. 17 is a functional diagram of a preferred embodiment of the inner rack structure of the present invention, wherein the rack structure comprises a lamp base with a plurality of UV-C light sources;

FIG. 18 is a functional diagram of the housing structure of an embodiment of the apparatus of the present invention, wherein the housing structure is removable;

FIG. 19 is a functional diagram of the housing structure of an embodiment of the apparatus of the present invention, wherein the housing structure comprises a door entry and a timer; and

FIG. 20 is a schematic of an interlock that prevents powering the light source or timer unless the housing enclosure is secured on the base, thereby preventing UV-C lamp activation unless the housing enclosure is installed;

FIG. 21 is a diagram showing E. coli bacteria recovered and grown on plates after exposure to UV-C irradiation for various time periods;

FIG. 22 is a graph showing decrease in colony forming units after exposure to UV-C light for various time periods;

FIG. 23A is a diagram showing microbial colonies grown from swabs of worn masks from different individuals;

FIG. 23B is a diagram comparing microbial colonies recovered and grown from masks before and after UV-C treatment; and

FIG. 23C is a diagram comparing microbial colonies recovered and grown from a mobile phone before and after UV-C treatment.

### DETAILED DESCRIPTION OF PREFERRED EMBODIMENTS

Referring now to the figures, wherein like reference numerals represent like parts throughout the several views, exemplary embodiments of the present disclosure will be described in detail. Throughout this description, various components may be identified having specific values, these values are provided as exemplary embodiments and should not be limiting of various concepts of the present invention as many comparable sizes and/or values may be implemented.

As will be described below, the present invention is capable of sterilizing multiple masks simultaneously, killing bacteria, yeasts, mold spores, and viruses, and may comprise different UV-C light sources (e.g., UV-C bulbs or UV-C light emitting diodes (LEDs), etc.) and may include a timer for a more reliable sterilization treatment.

With regard to the light source 4, it has been determined that a 110V bulb up to 60 watts is the most effective device for producing the UV-C light. These most conveniently can be sourced at 10 W, 15 W, 20 W, 25 W, 36 W, 38 W, 54 W, or 60 W bulb mercury or LED bulbs with an E26/E27 base, as well as others. As such, this eliminates the need for an E17 to E26/E27 adaptor or a transformer/capacitor. It is within the broadest scope of the present invention that the apparatus 1 may utilize either an E17 or E26/E27 bulb.

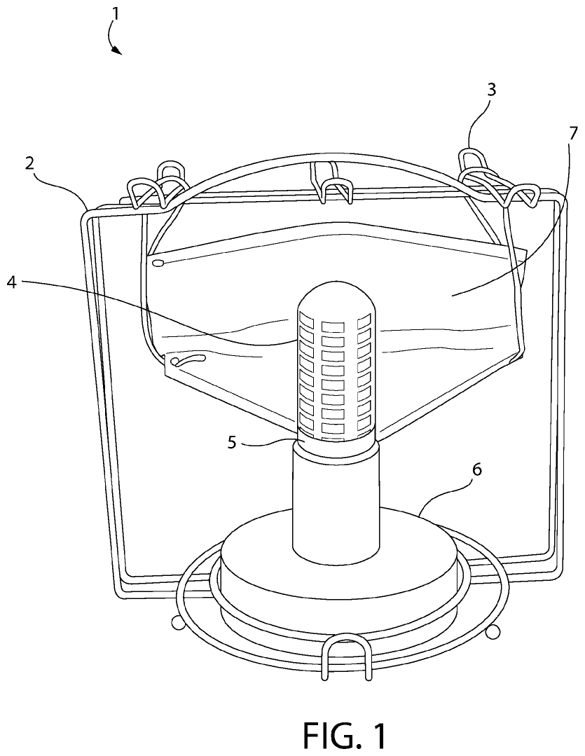

As shown in FIG. 1, the apparatus for UV-C mask sanitization 1 comprises a rack structure 2 comprising stainless steel; a plurality of arms 3 comprising stainless steel fasteners 10; and a UV-C light source 4 comprising a UV-C bulb which is releasably secured within a receptacle 5 on a base portion 6. By way of example only, as shown in FIG. 1, the receptacle 5 comprises an adaptor to an E26/E27 screw base and is centrally-positioned to deliver unobstructed and continuous UV-C radiation to the inner masks' surfaces. The preferred embodiment's bulb size is one which delivers quick and effective sanitizing properties with limited or no emission of ozone. Further, the light source 4 utilized in the preferred embodiment may comprise a low-pressure mercury bulb or a light emitting diode (LED) bulb with either an E26/E27 or E17 screw base. It should be noted that an adaptor is only used as part of the receptacle 5 for the embodiment of FIG. 1; all embodiments shown in the subsequent figures do not utilize an adaptor as part of the receptacle 5.

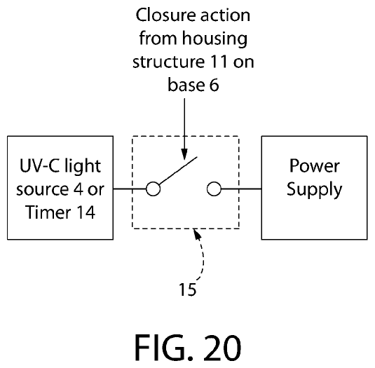

The present invention may also be referred to as a “portable hanging rack device” since it can be easily deployed anywhere in a household or workplace and involves “hanging” a plurality of contaminated masks on the rack structure 2. The rack structure 2 is constructed around a central UV-C light source 4 of 253.7 nm. As disclosed, the apparatus 1 is designed for personal use in the home or within the workplace for killing of airborne bacteria, viruses, yeast, and mold spores after 5 minutes or more of exposure to the UV-C light. The apparatus may be modified by increasing the strength or number of UV-C light sources to minimize the amount of sanitization time. As mentioned previously, the embodiment shown in FIG. 1 uses a UV-C bulb that produces limited ozone and whose receptacle 5/base portion 6 comprises an E26/E27 lamp screw base or a E17 screw base along with an adaptor to an E26/E27 lamp screw base. The bulb may need to be replaced every 6-8 months depending on level of use. Using the single bulb, the device may treat a plurality of contaminated masks (e.g., six masks) by positioning the masks vertically on the internal rack 2. Additionally, the present invention may be modified to allow for bulbs of different wattages/wavelengths with an E26/E27 base and may include a control timer 14 for easier and more precise use. To prevent the UV light from harming a user's skin and eyes, the light source 4 may be shielded with a housing enclosure 11. FIG. 20 provides an exemplary schematic of an interlock 15 that prevents powering the light source 4 or timer 14 unless the housing enclosure is secured on the base 6, thereby preventing UV-C light source 4 activation unless the housing enclosure 11 is installed.

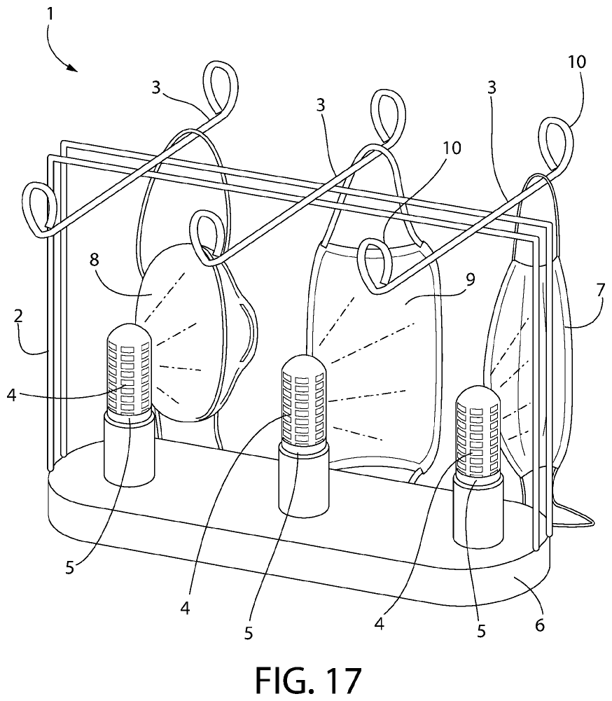

An alternative design of the present invention involves a tandem configuration whereby instead of “stacking” the contaminated masks vertically, the internal rack 2 comprises a horizontally-displaced series of light sources 4 and respective arms 3 for placing the contaminated masks thereon. In this alternative design, a plurality of UV-C light sources 4 is used to treat pairs of contaminated masks, as shown in FIG. 17. Although not shown, a corresponding housing enclosure is positioned over the tandem configuration and would also include the interlock 15 to prevent any of the light sources 4 from activating until the corresponding housing enclosure were positioned over the tandem configuration.

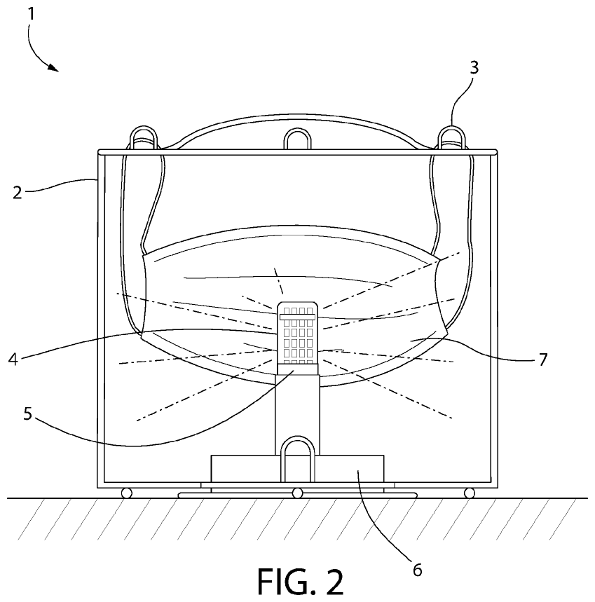

As shown in FIG. 2, the apparatus 1 for UV-C mask sanitization includes the rack structure 2 which comprises stainless steel and which comprises a plurality of arms 3 also comprising stainless steel fasteners; the apparatus 1 also comprises a UV-C light source 4 comprising a 110 volt UV-C bulb, wherein the bulb is also releasably securable within its receptacle 5 in the base portion 6 and is centrally-positioned to deliver unobstructed and continuous UV-C radiation to the inner mask surface. It should be noted that only a single mask 7 is depicted in FIGS. 1-2 for clarity. This UV-C light source 4 operates from standard household or workplace power. The preferred embodiment's bulb size is one which delivers quick and effective sanitizing properties without the significant emission of ozone. Further, the light source 4 utilized in the preferred embodiment may comprise a 110V low-pressure mercury bulb or a light emitting diode (LED) bulb. When UV-C radiation contacts the surgical mask 7 depicted in the preferred embodiment, the surface of the surgical mask 7 is subsequently sanitized as the radiation deactivates biological components of pathogens.

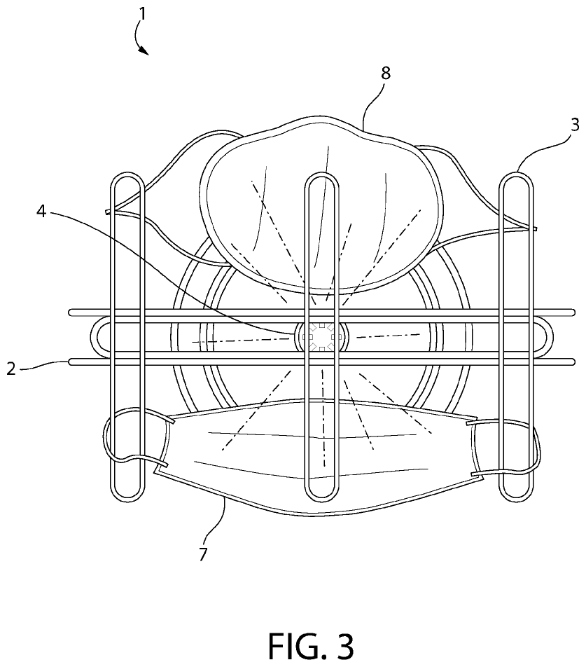

As shown in FIG. 3, the apparatus for UV-C mask sanitization 1 may comprise arms 3 on which the straps of an N-95 mask 8, surgical mask 7, or cloth mask 9 may hang. The arms 3 may be bent in an upward position to secure the mask in an expanded position. The preferred embodiment may also comprise a model that is able to hang two masks on either side of the UV-C light source 4, wherein the light source 4 is positioned to deliver uninterrupted UV-C radiation to the surface of both masks simultaneously.

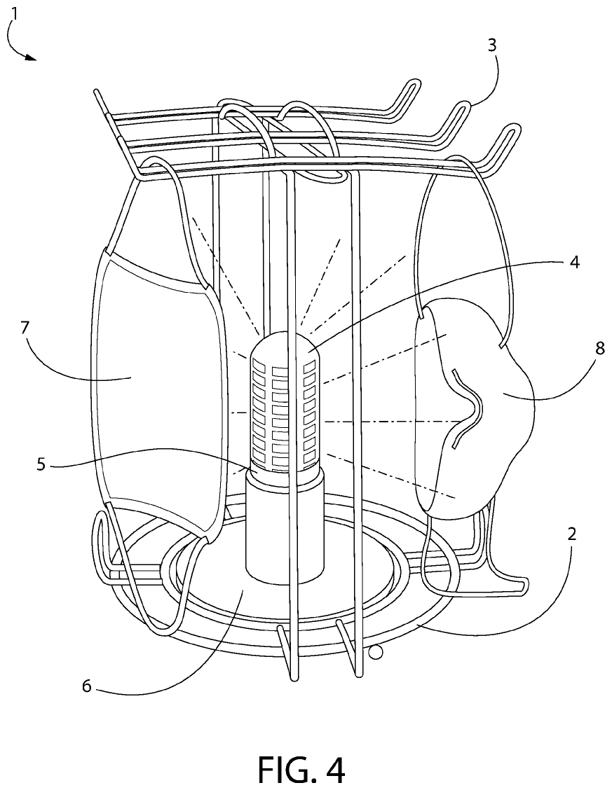

FIG. 4 is a side view of the preferred embodiment of the apparatus of FIG. 3 for UV-C mask sanitization 1, wherein the apparatus may utilize stainless steel arms 3 to hang both surgical masks 7 and N-95 masks 8 in a position where each may be thoroughly sanitized by the UV-C light source 4. The arms 3 depicted in the preferred embodiment may comprise stainless steel, aluminum, or another material which is capable of withstanding UV-C radiation.

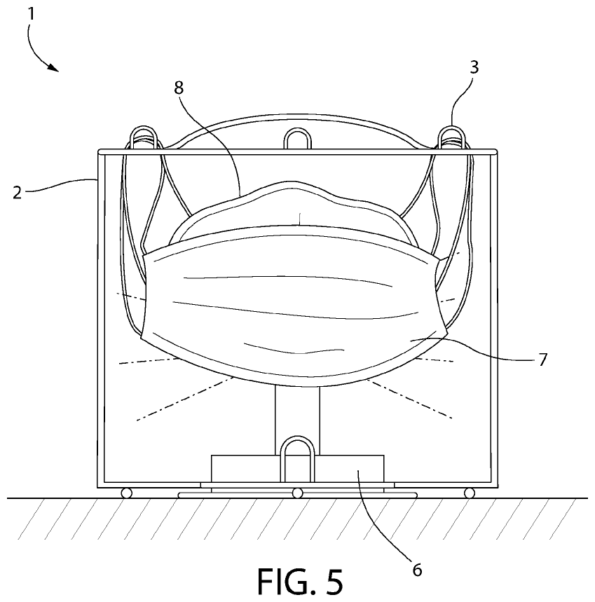

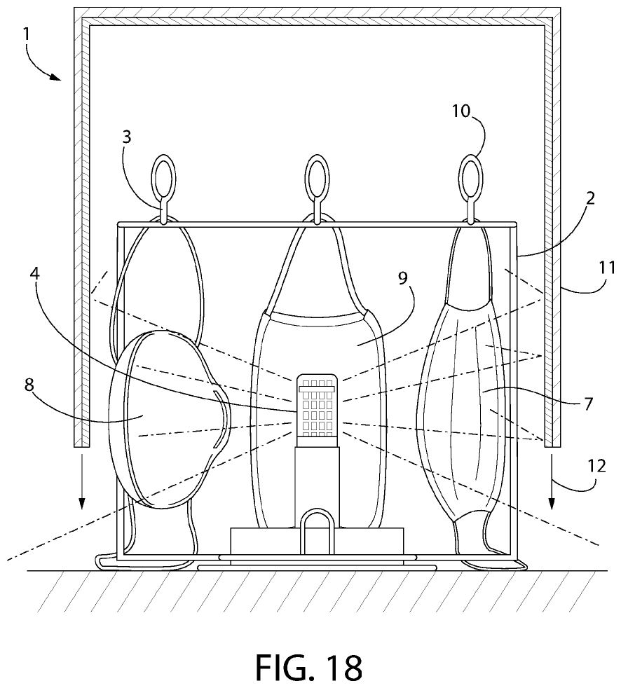

FIG. 5 depicts another side view of the preferred embodiment of the apparatus for UV-C mask sanitization 1 of FIG. 3, wherein the rack structure 2 is shown hanging both a surgical mask 7 and N-95 mask 8. The rack structure 2 may be enclosed within a housing structure 11 as depicted in FIG. 18 so as to prevent the escape of UV-C radiation from the apparatus during active sanitization.

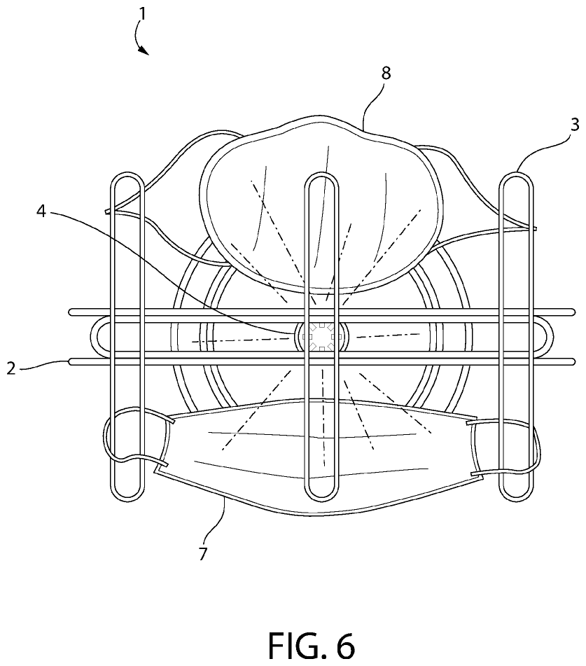

FIG. 6 depicts a top view of a preferred embodiment of the apparatus for UV-C mask sanitization 1, wherein the arms 3 serve as attachment points for a surgical mask 7 and N-95 mask 8. As depicted in the preferred embodiment, the UV-C light source 4 delivers uninterrupted UV-C radiation to the mask surfaces.

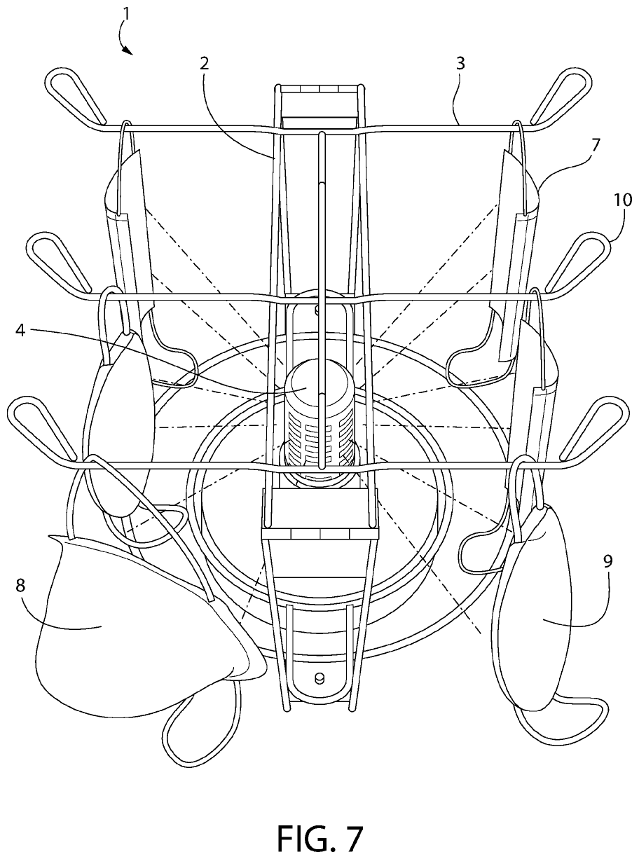

FIG. 7 depicts a top view along the width of a preferred embodiment of the apparatus for UV-C mask sanitization 1, wherein the embodiment comprises six arms 3, wherein each arm 3 comprises a fastener 10 to secure a mask in an expanded position. The fastener 10 may comprise a hook, clip, or other securing mechanism. Further, each arm 3 depicted in the preferred embodiment is capable of holding a mask in an expanded position. While the preferred embodiment may comprise arms 3 capable of holding six masks, more arms 3 may be added to hold additional masks. While the prototype utilizes a 110 V UV-C bulb, the bulb size and type may be changed or additional bulbs may be added to provide adequate radiation levels for sanitization of more than six masks without producing ozone.

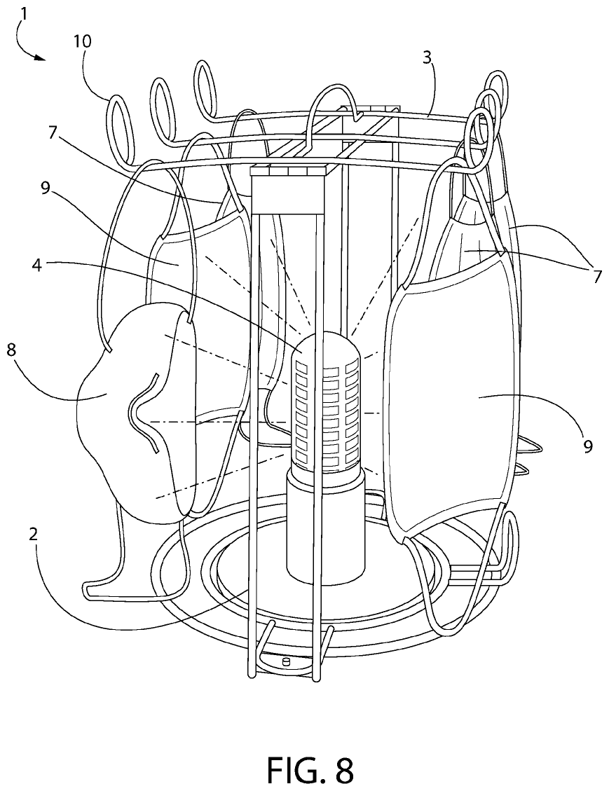

FIG. 8 depicts a side view along the width of a preferred embodiment of the rack structure 2, wherein the apparatus 1 is capable of sanitizing surgical masks 7, N-95 masks 8, and cloth masks 9.

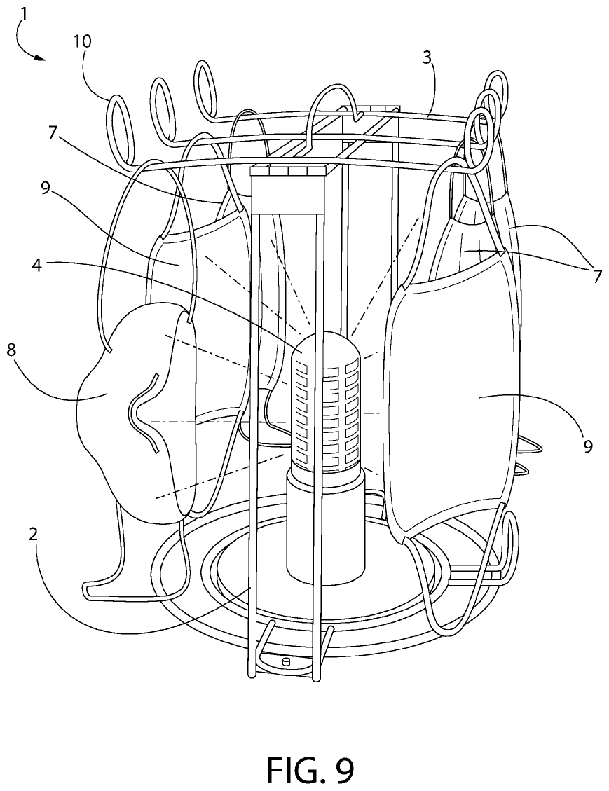

FIG. 9 depicts another side view along the width of a preferred embodiment of the rack structure 2, wherein the apparatus 1 is capable of sanitizing surgical masks 7, N-95 masks 8, and cloth masks 9.

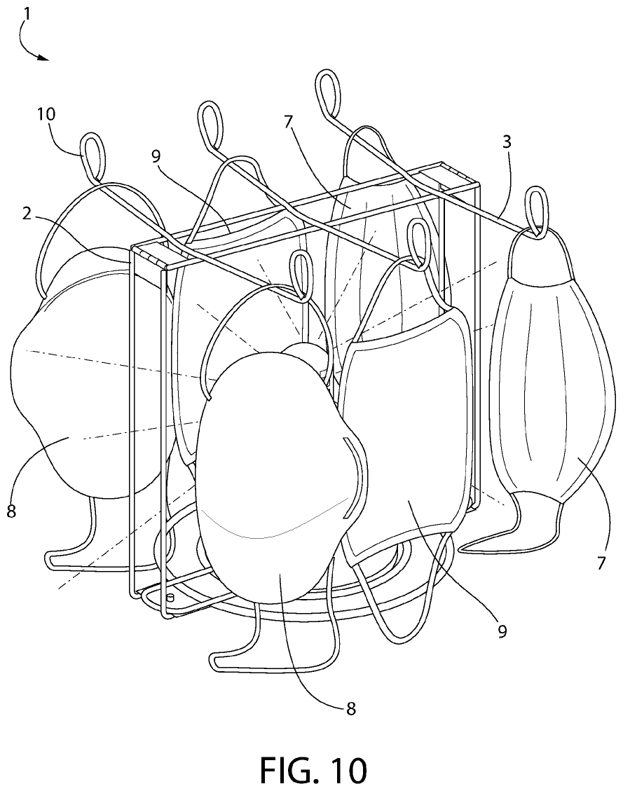

FIG. 10 depicts an angled top-view of the preferred embodiment of the rack structure 2 of the apparatus 1, wherein each mask is hung vertically from each arm 3 of the structure. Masks may also be positioned horizontally within the rack structure 2, wherein the fasteners 10 on each arm secure a portion of the mask. The arms 3 comprise a fastener 10 in the form of a hook at the end of each arm 3, wherein the fastener 10 prevents the mask from falling off of the rack structure 2.

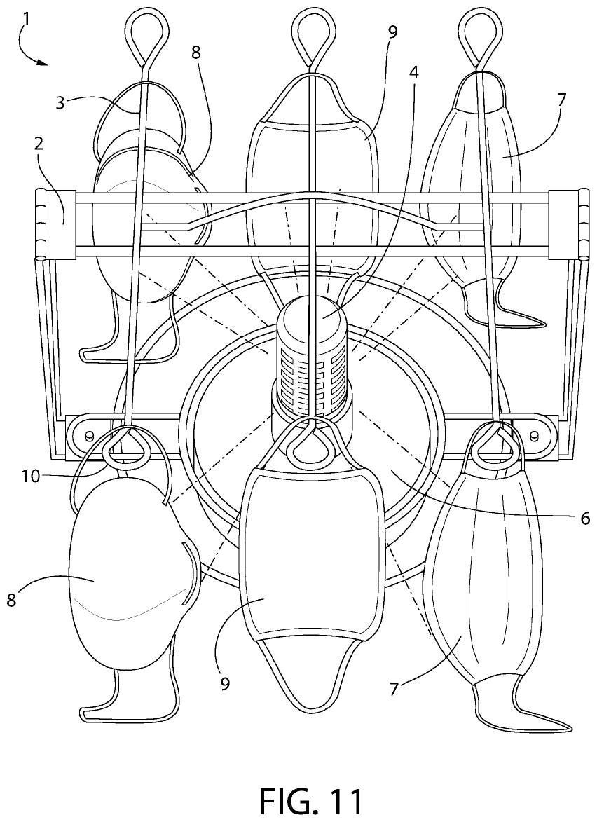

FIG. 11 depicts a top-view of the preferred embodiment of the rack structure 2 of the apparatus 1, wherein the apparatus 1 is capable of holding six masks. Each arm 3 may hold a mask in an expanded position without causing overlap with an adjacent mask, enabling UV-C radiation to reach the entire inner surface of each mask.

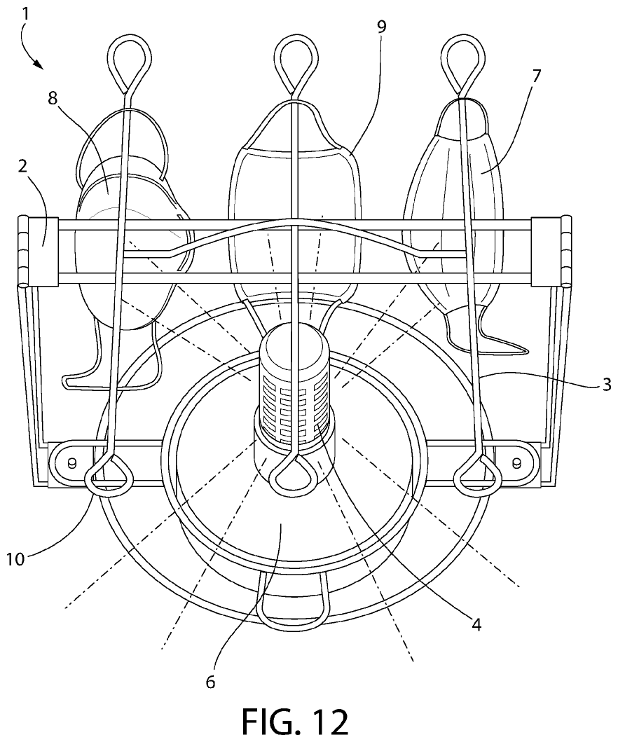

FIG. 12 depicts a top-view of the preferred embodiment of the rack structure 2 of the apparatus 1 but with three of the contaminated masks removed for clarity. The preferred embodiment may comprise six arms 3 to hang masks but may be modified to include additional arms 3. The arms 3 may comprise fasteners 10, which may comprise hooks, clips, or other mechanisms to which masks or other small household objects, such as keys, may be secured.

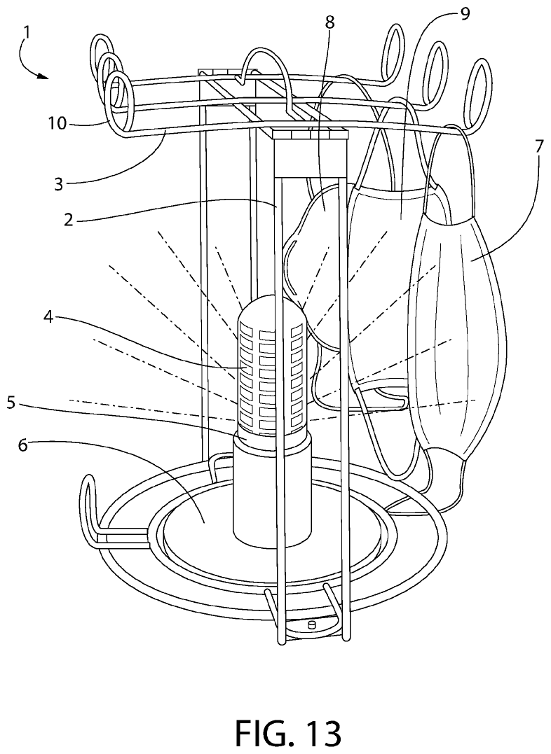

FIG. 13 depicts a side-view along the width of the preferred embodiment of the rack structure 2 of the apparatus 1 of FIG. 12. The UV-C light source 4 utilized in the preferred embodiment comprises a 110 V bulb so as to enable the bulb to connect to the lamp base 6 via a screw-in mechanism. While the bulb may comprise a larger size or an LED light source 4, the preferred embodiment's bulb size enables it to attach to the receptacle 5 without the need of a transformer. As transformers, particularly those with pins and a double-holder attachment mechanism, would increase the bulkiness of the UV-C light source, maintaining a bulb size capable of a screw-in mechanism maintains a compact and cost-effective UV-C light source.

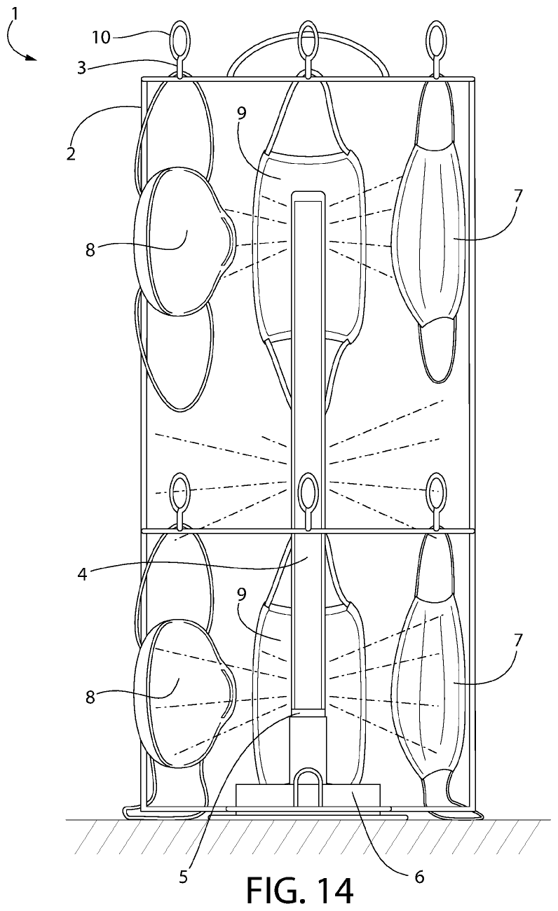

FIG. 14 depicts a side view along the length of the preferred embodiment of the rack structure 2 of the apparatus 1. The rack structure 2 may comprise a single tier or multiple tiers (e.g., vertically-displaced), wherein each tier comprises arms 3 which may comprise fasteners 10 to secure surgical masks 7, N-95 masks 8, or cloth masks 9. In a multiple-tier rack structure, the UV-C light source 4 may comprise a bulb, a tube light source, or multiple bulbs to deliver adequate light to multiple tiers without the production of ozone. Further, in a single tier rack structure, the rack structure may be modified from a single-bulb structure to include multiple UV-C light sources equally dispersed along the length of the apparatus 1 as depicted in FIG. 17, wherein each UV-C light source 4 is centrally located amongst arms 3 on the rack structure which accommodate additional masks.

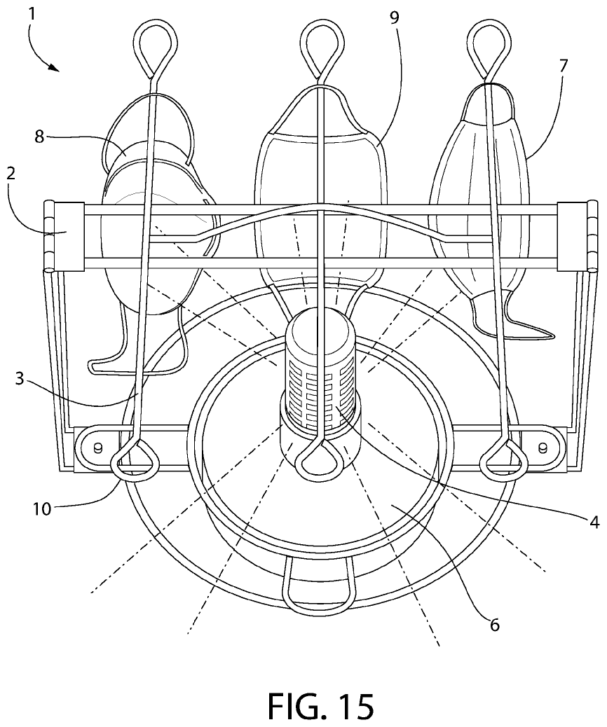

FIG. 15 depicts a top-view of the preferred embodiment of the rack structure 2 of the apparatus 1, wherein the rack structure 2 may accommodate surgical masks 7, N-95 masks 8, and cloth masks 9. The rack structure 2 of the preferred embodiment is designed to accommodate multiple masks to enable use by an entire family, while also maintaining a compact, portable size rendering it capable of use in the home or workplace.

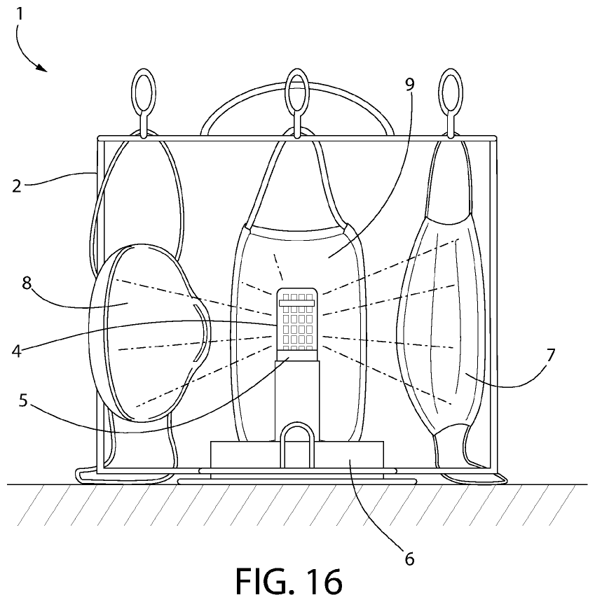

FIG. 16 depicts a side-view along the length of the preferred embodiment of the rack structure 2 of the apparatus 1, wherein the UV-C light source 4 delivers UV-C radiation to the inner surfaces of surgical masks 7, N-95 masks 8, cloth masks 9, and other face coverings.

FIG. 17 is a functional diagram of the internal rack structure 2 of the preferred embodiment of the present invention, wherein the rack structure 2 is attached to an elongated lamp base 6, wherein the lamp base 6 provides for a plurality of UV-C light sources 4 and receptacles 5, previously referred to the “tandem configuration”. The use of a plurality of UV-C light sources 4 enables the rack structure 2 to accommodate a plurality of arms 3 and fasteners 10 to accommodate a plurality of masks at a single time. This embodiment enables larger families or other groups of individuals to sanitize a greater number of masks at once without the need of an industrial-sized apparatus.

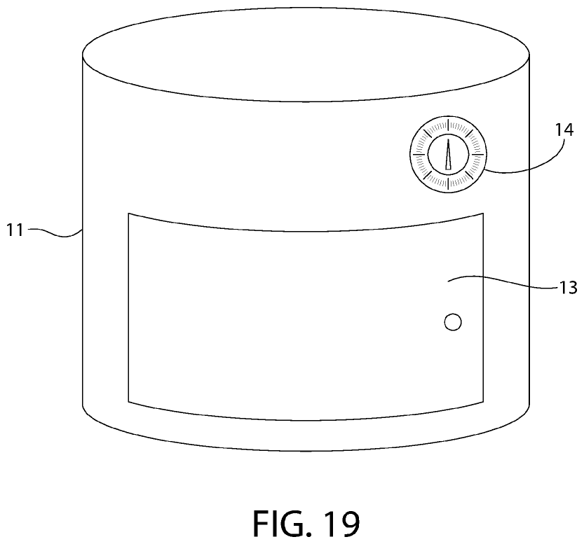

FIG. 18 is a functional diagram of the housing structure 11 of the preferred embodiment, wherein the detachable housing structure 12 completely covers the inner rack structure 2 of the apparatus and prevents UV-C radiation from escaping the apparatus 1. The inner surface of the housing structure 11 may comprise aluminum, stainless steel, or another reflective metallic surface capable of both withstanding UV-C radiation and reflecting UV-C light throughout the inside of the apparatus 1. The reflective surface enables UV-C radiation to reach all surfaces of the masks and optimizes sanitization. The housing structure 11 may further comprise a detachable structure 12, wherein users may lift the housing structure 11 off of the rack structure 2 to gain access to the inner rack structure 2, or a door 13, wherein the door 13 may open and grant users access to the inside of the housing structure 2 as shown in FIG. 19.

FIG. 19 is a functional diagram of the housing structure of a preferred embodiment, wherein the housing structure 11 comprises a door entry 13 and a timer 14. The timer 14 controls the length of UV-C radiation exposure to optimize sanitization while preventing over-exposure of UV-C radiation to the fibers of the masks. The timer 14 may further comprise a feature to alert users when sanitization is complete, wherein the feature may comprise an alert tone or visual display. The timer 14 may further comprise an analog or digital structure, wherein users may start the timer by turning a dial or pushing a button to initiate the sanitization process. Such a timer 14 may be utilized on housing structures 11 utilizing a door entry 13 or those that are detachable structures 12 as depicted in FIG. 18.

It is also within the broadest scope of the present invention to include other types of small personal items (e.g., keys, cell phones, money, credit cards, mail, etc.) that may be placed on/in the present invention for sanitizing with the UV light treatment method.

### EXAMPLES

An apparatus in accordance with examples of the invention was used with a UV-C wavelength of approximately 254 nm, which has the ability to kill bacteria, viruses, yeast, and mold spores. A housing structure (FIGS. 18 & 19) was used to protect users from the potentially damaging effects of UV-C exposure. XL-1 Blue E. coli (Stratagene) were inoculated in Lysogeny Broth (LB) and grown overnight at 37° C. in a shaking incubator and then diluted 1:500 in LB. Multiple droplets of 5u1 of E. coli were spotted approximately 1 cm apart across the inside surface (face-side) of surgical masks and incubated for 10 minutes at room temperature. The surgical masks were then hung in the apparatus and the inside surfaces were exposed to UV-C light at a measured density of 0.8 mW/cm2 at a distance of 10 cm from the light source for 0 (control), 30, 60 or 120 seconds. A sterile swab was used to recover surviving bacteria and streaked across the surface of LB plates and allowed to air dry. The plates were incubated overnight at 37° C., and colonies were counted after 24 hours to calculate time kill curves from samples in quintuplicate and the point of a 3 log 10-fold decrease in Colony Forming Units (CFUs), which represents 99.9% sanitization. Pairwise t-tests assuming equal variances were performed between untreated controls and that of each time point to calculate p-values. To assess sanitization of environmental microorganisms on face masks, cloth and surgical masks that had been worn for at least one day were evaluated. The inside surface of each mask was swabbed prior to UV-C exposure or after 5 minutes of UV-C treatment, as described above. The swab was streaked across the surface of LB plates, grown for 24 hours at 37° C., and then photographed. Similarly, a swab was taken from the glass touch screen of a mobile phone prior to UV-C exposure and after 5 minutes of UV-C treatment.

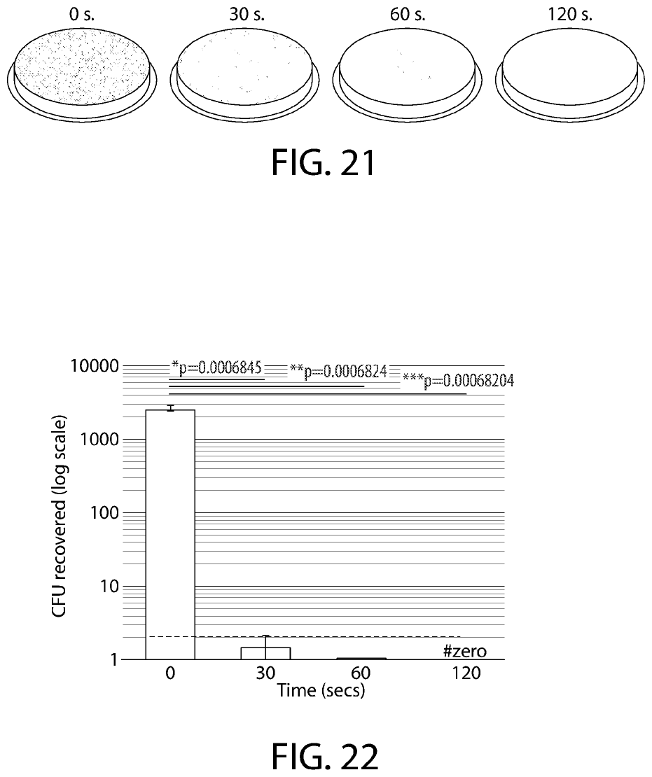

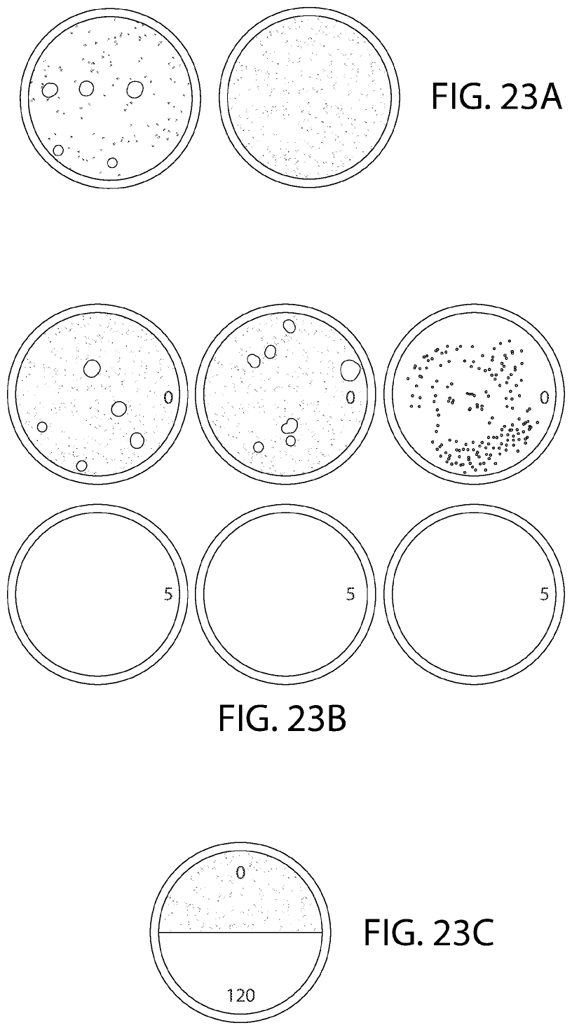

To test the efficiency of UV-C light in the sanitization of face masks, an embodiment of the invention capable of simultaneously hanging multiple masks or other personal items such as keys or cell phones was used (FIG. 14). E. coli were purposely spotted in droplets on the inner mask surface and then exposed to UV-C irradiation for various times (0, 30, 60, and 120 seconds), and surviving bacteria were recovered and grown on LB plates (FIG. 21). CFUs were counted and compared to untreated controls. UV-C treatment of masks for 30 seconds led to a greater than 3 log 10-fold decrease in colony forming units, representing a 99.94% killing of bacteria, p=0.0006845 (FIG. 22). Efficiency of killing reached 99.99% after 1 minute (p=0.0006824), and no CFUs were recovered after a treatment of 2 minutes (p=0.00068204). The ability of the apparatus to sanitize donated soiled cloth or surgical face masks that had been exposed to environmental microorganisms after being worn for a full day was also tested. A variety of microbial colonies grew from swabs of worn masks from different individuals, including large colonies characteristic of Bacillus species (FIG. 23A). Swabs were also taken from the internal face-side of each mask prior to UV-C exposure and following 5 minutes of treatment measured at 0.8 mW/cm2 at a distance of 10 cm from the light source. Samples were then streaked on LB plates and incubated for 24 hours. The results demonstrated that numerous microbial colonies grew from swabs of both the worn cloth and surgical masks (FIG. 23B—top row) and that no CFUs were recovered following UV-C treatment of the same masks (FIG. 23B—bottom row). Finally, the ability of the invention to sanitize the touch screen of a mobile phone, which is another possible source of transmissible microorganisms, was tested. A swab taken from the mobile phone and transferred to an LB plate led to the growth of a number of microbial CFUs within 24 hours, albeit fewer than observed from masks (FIG. 23C, upper panel). Swabs of the phone following treatment with UV-C light for 5 minutes, as above, revealed that no CFUs emerged (FIG. 23C, lower panel).

While the invention has been described in detail and with reference to specific examples thereof, it will be apparent to one skilled in the art that various changes and modifications can be made therein without departing from the spirit and scope thereof.

### REFERENCES

1. Sharma, N. M., Chaudhary, A. R. (2020). Evolution of Masks As Public Health Intervention in the Control of Respiratory Outbreaks. National Journal of Community Medicine, 11(3), 138-140.
2. Balazs, P., Foley, K. L. (2010). The Austrian Success of Controlling Plague in the 18th Century: Maritime Quarantine Methods Applied to Continental Circumstances. Kaleidoscope: Journal of History, Culture, & Medicine, 1(1), 73-87.
3. Rubio-Romero, J. C., Pardo-Ferreira, M. C., Torrecilla-Garcia, J. A., et al. (2020). Disposable Masks: Disinfection and Sterilization for Reuse, and Non-certified Manufacturing, in the Face of Shortages During the COVID-19 Pandemic. Safety Science, 129, 1-11: 104830.
4. Leung, N. H. L., Chu, D. K. W., Shiu, E. Y. C., et al. (2020). Respiratory Virus Shedding in Exhaled Breath and Efficacy of Face Masks. Nature Medicine, 26, 676-680.
5. Brosseau, L. M., McCullough, N. V., Vesley, D. (1997). Bacterial Survival on Respirator Filters and Surgical Masks. Journal of the American Biological Safety Association, 2(3) 32-43.
6. How to Wash Masks (2020). Center for Disease Control, https://www.cdc.gov/coronavirus/2019-ncov/prevent-getting-sick/how-to-wash-cloth-face-coverings.html.
7. How to Wear Masks (2020). Center for Disease Control, https://www.cdc.gov/coronavirus/2019-ncov/prevent-getting-sick/how-to-wear-cloth-face-coverings.html.
8. Reed, N. G. (2010). The History of Ultraviolet Germicidal Irradiation for Air Disinfection. Public Health Reports. 25 (January-February 2010), 15-27.
9. Casini, B., Tuvo, B. Cristina, M. L., et al. (2019). Evaluation of an Ultraviolet C (UVC) Light-Emitting Device for Disinfecton of High Touch Surfaces in Hospital Critical Areas. International Journal of Environmental Research ad Public Health, 16, 3572. 1-10.
10. Ozone Generators that are Sold as Air Cleaners, United States Environmental Protection Agency, https://www.epa.gov/indoor-air-quality-iaq/ozone-generators-are-sold-air-cleaners#:˜:text=When%20inhaled%2C%20ozone%20can%20damage,body%20to%20fight%20respiratory%20infections.
11. Orazio, J. D., Jarrett, S., Amaro-Ortiz, A., et al. (2013). UV Radiation and the Skin. International Journal of Molecular Sciences, 14, 122222-12248.
12. Behar-Cohen, F., Baillet, G., Ayguavives, T. (2014). Ultraviolet Damage to the Eye Revisited: Eye-Sun Protection Factor (E-SPF®), a New Ultraviolet Protection Label for Eyewear, Clinical Ophtalmology. 8, 87-104.
13. Byrns, G., Barham, B., Yang, L., et al. (2017). The Uses and Limitations of a Hand-Held Germicidal Ultraviolet Wand for Surface Disinfection. Journal of Occupational and Environmental Hygiene. 14(10), 749-57.
14. Hadi, A. G., Yousif, E., El-Hiti, G. A., et al. (2019). Long-Term Effect of Ultraviolet Irradiation on Poly(vinyl chloride) Films Containing Naproxen Diorganotin (IV) Complexes. Molecules, 24, 2396.

1. An apparatus for UV-C sanitization of contaminated masks in a household or workplace environment comprising:
a lamp base;
a rack structure, wherein the rack structure is coupled to the lamp base;
a plurality of arms, wherein the plurality of arms is coupled to the rack structure;
a UV-C light source wherein said lamp base, said UV-C light source,
said rack structure and plurality of arms form an assembly; and
a housing structure that is configured to cover said assembly for preventing UV-C radiation from escaping the apparatus.

2. The apparatus of claim 1, wherein the plurality of arms comprises fasteners.

3. (canceled)

4. The apparatus of claim 1, wherein the UV-C light source is centrally located amongst the plurality of arms.

5. (canceled)

6. The apparatus of claim 1, wherein the UV-C light source comprises a low-pressure mercury bulb or a light emitting diode (LED) bulb.

7. The apparatus of claim 1, wherein the UV-C light source comprises a 110 V UV-C bulb, wherein the 110 V UV-C bulb is coupled to an E26/27 screw base or an E17 screw base.

8. (canceled)

9. (canceled)

10. (canceled)

11. (canceled)

12. The apparatus of claim 1, wherein the UV-C light source is detachably secured to a receptacle.

13. The apparatus of claim 12, wherein the receptacle comprises an adaptor.

14. The apparatus of claim 13, wherein the adaptor corresponds to an E26/27 screw base.

15. The apparatus of claim 1, wherein the housing structure comprises an inner surface comprising aluminum, stainless steel, or another reflective material capable of withstanding UV-C radiation without damage to the material's structural integrity.

16. The apparatus of claim 1, wherein the housing structure detaches from the rack structure.

17. The apparatus of claim 1, wherein the housing structure comprises a door or other entryway to give a user access to the rack structure.

18. The apparatus of claim 1, wherein the housing structure comprises a timer.

19. The apparatus of claim 18, wherein the timer deactivates the UV-C light source when sanitization is complete.

20. The apparatus of claim 18, wherein the timer alerts users when sanitization is complete.

21. (canceled)

22. (canceled)

23. The apparatus of claim 1, wherein the housing structure comprises an interlock device, wherein the interlock device prohibits activation of the UV-C light source unless the housing structure is covering said assembly.

24. A method for UV-C sanitization of contaminated masks in a household or workplace environment, said method comprising:
providing a rack structure, configured for holding a plurality of contaminated masks, with a UV-C light source positioned within said rack structure;
covering said rack structure with a housing structure to reflect UV-C radiation that emanates from said UV-C light source, when activated, while also protecting any one in a vicinity of said UV-C light source from exposure to the UV-C radiation;
activating said UV-C light source to deliver continuous UV-C radiation for a predetermined period towards said plurality of contaminated masks in order to sanitize said plurality of contaminated masks;
deactivating said UV-C light source; and
providing access to sanitized masks via said housing structure.

25. The method of claim 24, wherein each mask of said plurality of masks is secured to the arms of the rack structure with a fastener comprising a hook, clip, or other securing mechanism.

26. The method of claim 25, wherein respective fasteners are attached to the mask on the mask's superior and inferior ends to hold the mask in an expanded position.

27. (canceled)

28. The method of claim 24, wherein said step of activating said UV-C light source comprises providing a timer coupled to said UV-C light source that can be set to activate said UV-C light for said predetermined period.

29. The method of claim 24, wherein said step of activating said UV-C light source comprises interlocking power provided to said UV-C light source, said interlocking power requiring that said housing structure is covering said UV-C light source and said rack structure before said UV-C light is activated.

30. (canceled)
